(19) United States
(12) Patent Application Publication
(10) Pub. No.: US 2025/0082577 A1
(43) Pub. Date: Mar. 13, 2025

(54) POLYMER BEADLET COMPOSITIONS AND METHODS OF USE THEREOF FOR IMPROVED STABILITY, BIOAVAILABILITY & SUSTAINED RELEASE

(71) Applicant: SpecNova LLC, Boca Raton, FL (US)

(72) Inventor: Sebastian Balcombe, Evergreen, CO (US)

(73) Assignee: SpecNova LLC, Boca Raton, FL (US)

(21) Appl. No.: 18/885,217

(22) Filed: Sep. 13, 2024

Related U.S. Application Data
(60) Provisional application No. 63/582,428, filed on Sep. 13, 2023.

(51) Int. Cl. A61K 9/16; A61K 9/20; A61K 9/50

(52) U.S. Cl. CPC A61K 9/1652; A61K 9/2054; A61K 9/5047

(57) ABSTRACT

A composition for sustained release of an active comprising: a plurality of beadlets, wherein each of the plurality of beadlets comprises: a polymer beadlet shell, wherein the active is dispersed within the polymer beadlet shell, the polymer beadlet shell comprises: gum arabic; microcrystalline cellulose (MCC); hydroxypropyl methylcellulose (HPMC); glycerol; γ-cyclodextrin; ethyl cellulose; and one or more metal halide salts.

### CROSS-REFERENCE TO RELATED APPLICATION(S)

This application claims priority to U.S. Provisional Application No. 63/582,428 filed Sep. 13, 2024, and entitled “POLYMER BEADLET COMPOSITIONS AND METHODS OF USE THEREOF FOR IMPROVED STABILITY, BIOAVAILABILITY & SUSTAINED RELEASE,” which is hereby incorporated by reference in its entirety under 35 U.S.C. § 119(e).

### BACKGROUND

The low bioavailability of many dietary supplements presents a challenge to realizing full potential efficacy. Conversely, the extremely high absorption of some dietary supplements presents a problem when the aim of supplementation is to achieve a sustained and stable increase of the supplement in the body. Rapid spikes in supplement blood levels can exceed the capacity of key transporters to deliver supplements to their cellular targets. Therefore, there is a need in the art for improved compositions and methods to achieve sustain/controlled release of dietary supplements.

### BRIEF SUMMARY

Disclosed herein are various embodiments of a system and related methods directed to the sustained release of an active as may be achieved through various compositions. In certain embodiments, the composition comprises a plurality of beadlets. In certain embodiments, each beadlet of the plurality of beadlets comprises a polymer beadlet shell and the active is dispersed within the polymer beadlet shell. According to certain embodiments, the polymer beadlet shell comprises: Gum Arabic; Microcrystalline cellulose (MCC), Hydroxypropyl methylcellulose (HPMC); glycerol, γ-cyclodextrin, ethyl cellulose; and/or one or more metal halide salt.

In certain embodiments, gum Arabic comprises about 1-25% of the polymer beadlet shell.

In certain embodiments, MCC comprises about 1-5% of the polymer beadlet shell.

In certain embodiments, HPMC comprises about 1-5% of the polymer beadlet shell.

In certain further embodiments, glycerol comprises about 1-3% of the polymer beadlet shell.

In yet further embodiments, γ-cyclodextrin comprises about 1-3% of the polymer beadlet shell. In still further embodiments, ethyl cellulose comprises about 1-5% of the polymer beadlet shell.

According to certain embodiments, the one or more metal halide salt comprises sodium chloride. In certain further embodiments, the one or more metal halide salt further comprises potassium chloride. In yet further embodiments, one or more metal halide salt further comprises magnesium chloride. In certain implementations of any of the foregoing embodiments, the one or more metal halide salt comprises from about 0.25-1% of the polymer beadlet shell.

According to certain embodiments, each of the plurality of beadlets has a diameter of from about 500 to 2000 μm.

In certain embodiments, actives suitable for sustained release by the instantly disclosed composition may be any active known in the art for which a sustained/slow release is desirable. In certain embodiments, one or more active is selected from beta-alanine, probiotics, caffeine, Berberine and melatonin. In further embodiments, the active is creatine. In certain embodiments, creatine is in the form of creatine monohydrate. In further embodiments, creatine is in the form of creatine HCl.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows a curve of creatine monohydrate release from a creatine monohydrate beadlet, according to certain embodiments.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. All percentages are weight percentages (wt %), unless otherwise indicated.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles” would either completely lack particles, or so nearly completely lack particles that the effect would be the same as if it completely lacked particles. In other words, a composition that is “substantially free of an ingredient or element may still actually contain such an item as long as there is no measurable effect thereof.

Admixing or admixed means the formation of a physical combination of two or more elements which may have a uniform or non-uniform composition throughout and includes, but is not limited to, solid mixtures, solutions and suspensions.

Aqueous and aqueous solution mean that water is present but does not require that water be the predominant component. For purposes of illustration and not in limitation, a solution of 90 volume percent of ethylene glycol and 10 volume percent water would be an aqueous solution. Aqueous solutions include liquid media containing dissolved or dispersed components such as, but not in limitation, colloidal suspensions and slurries.

As used herein, “active” or “bioactive” means the compound in the supplement that is responsible for producing the intended health benefit or therapeutic effect. This “active” component is typically the primary substance that contributes to the supplement's efficacy, such as vitamins, minerals, herbs, amino acids, enzymes, or other nutrients. In certain embodiments, one or more active may be dispersed within the instantly disclosed beadlets.

The present disclosure relates to a novel polymer beadlet formulation and a novel preparation process where certain actives (e.g., dietary supplements and/or probiotic) bioavailability is increased through allowing a slow release of the bioactive over 8-24 hours, greatly improving the bioactive's stability in the body, allowing more of the bioactive to be available for absorption at a targeted location within the body (e.g., the small intestine). The slow/controlled release of certain bioactives can provide significant benefit. For example, where a dietary ingredient absorbs into the blood too fast and overwhelms/oversaturates the ability of the target cells to transport and/or uptake it, significant quantities of the bioactive are excreted rather than reaching and being absorbed into its target tissue.

Examples include creatine or the non-essential amino-acid beta-alanine, which absorb rapidly into the blood. In the case of creatine, the CREAT1 transporter can only transport creatine intracellularly at a known rate, and the CREAT1 transporter ends up down regulating with high levels of creatine in the blood plasma as a result. Similarly, the beta-alanine transporter TauT (taurine transporter—SLC6A6) can become over saturated with high blood plasma levels of beta-alanine or PAT1(SLC36A1). As would be understood, taking small 800 mg doses of beta-alanine 4 times a day is more effective at increasing intracellular carnosine levels than taking a bolus of 3.3 g a day.

Disclosed herein are various embodiments of a system and related methods directed to the sustained release of an active as may be achieved through various compositions. In certain embodiments, the composition comprises a plurality of beadlets. In certain embodiments, each beadlet of the plurality of beadlets comprises a polymer beadlet shell and the active is dispersed within the polymer beadlet shell. According to certain embodiments, the polymer beadlet shell comprises: Gum Arabic; Microcrystalline cellulose (MCC), Hydroxypropyl methylcellulose (HPMC); glycerol, γ-Cyclodextrin, ethyl cellulose; and/or one or more metal halide salt.

In certain embodiments, Gum Arabic comprises about 1-25% of the polymer beadlet shell.

In certain embodiments, MCC comprises about 1-5% of the polymer beadlet shell.

In certain embodiments, HPMC comprises about 1-5% of the polymer beadlet shell.

In certain further embodiments, glycerol comprises about 1-3% of the polymer beadlet shell.

In yet further embodiments, γ-cyclodextrin comprises about 1-3% of the polymer beadlet shell.

In still further embodiments, ethyl cellulose comprises about 1-5% of the polymer beadlet shell.

According to certain embodiments, the one or more metal halide salt comprises sodium chloride. In certain further embodiments, the one or more metal halide salt further comprises potassium chloride. In yet further embodiments, one or more metal halide salt further comprises magnesium chloride. In certain implementations of any of the foregoing embodiments, the one or more metal halide salt comprises from about 0.25-1% of the polymer beadlet shell.

According to certain embodiments, each of the plurality of beadlets has a diameter of from about 500 to 2000 μm.

In certain embodiments, actives suitable for sustained release by the instantly disclosed composition may be any active known in the art for which a sustained/slow release is desirable. In certain embodiments, one or more active is selected from Beta-Alanine, probiotics, caffeine, Berberine and melatonin. In further embodiments, the active is creatine. In certain embodiments, creatine is in the form of creatine monohydrate. In further embodiments, creatine is in the form of creatine HCl.

In certain implementations, upon ingestion of the compositions disclosed herein, the active is released over a period of from about 10-12 hours.

In certain embodiments, the polymer beadlet shell further comprises a natural colorant. In exemplary implementations, the natural colorant is selected from paprika extract, turmeric, beta-carotene, and beet extract. In certain implementations, the natural colorant comprises from about 0.25 to 2.0% of the polymer beadlet shell.

In certain embodiments, the instantly disclosed beadlets enhance subject compliance with a dosing regimen, where a single dose of a dietary ingredient can be taken, rather than multiple times a day.

The instantly disclosed composition can be used to deliver numerous actives, including, but not limited to creatine (all forms), Beta-Alanine, probiotics, stimulants such as caffeine when one is looking for a sustained release effect, berberine (e.g., to achieve longer lasting glucose control), and/or melatonin (which has a very short half-life).

Further disclosed herein are methods for providing sustained release of an active to a subject comprising administering to the subject a composition disclosed herein.

Various aspects and embodiments of the present disclosure are defined by the following numbered clauses:
1. A composition for sustained release of one or more active comprising:
a plurality of beadlets, wherein each of the plurality of beadlets comprise:
a polymer beadlet shell, wherein the active is dispersed within the polymer beadlet shell, the polymer beadlet shell comprises: gum arabic; microcrystalline cellulose (MCC); hydroxypropyl methylcellulose (HPMC); glycerol; γ-cyclodextrin; ethyl cellulose; and one or more metal halide salt.
2. The composition of clause 1, wherein the Gum Arabic comprises from about 1-25 wt % of the polymer beadlet shell.
3. The composition of clause 2, wherein MCC comprises about 1-5 wt % of the polymer beadlet shell.
4. The composition of clause 3, wherein HPMC comprises about 1-5 wt % of the polymer beadlet shell.
5. The composition of clause 1, wherein glycerol comprises about 1-3 wt % of the polymer beadlet shell.
6. The composition of clause 1, wherein the γ-cyclodextrin comprises about 1-3 wt % of the polymer beadlet shell.
7. The composition of clause 1, wherein the ethyl cellulose comprises about 1-5 wt % of the polymer beadlet shell.
8. The composition of clause 1, wherein the one or more metal halide salt comprises sodium chloride.
9. The composition of clause 8, wherein one or more metal halide salt further comprises potassium chloride.
10. The composition of clause 9, wherein one or more metal halide salt further comprises magnesium chloride.
11. The composition of any of clauses 8-10, wherein the one or more metal halide salt comprises from about 0.25-1 wt % of the polymer beadlet shell.
12. The composition of clause 1, wherein one or more active is selected from Beta-Alanine, Probiotics, caffeine, Berberine and melatonin.
13. The composition of clause 12, wherein the active is creatine.
14. The composition of clauses 12-13, wherein the active is released over a period of from about 10-12 hours, following oral consumption.
15. The composition of clause 1, where each of the plurality of beadlets has a diameter of from about 500 to 2000 μm.
16. The composition of any preceding clause, where the polymer beadlet shell further comprises a natural colorant.
17. The composition of clause 16, the natural colorant is selected from paprika extract, turmeric, beta-carotene, and beet extract.
18. The composition of clause 17, wherein the natural colorant comprises from about 0.25 to 2.0 wt % of the polymer beadlet shell.
19. A composition for sustained release of creatine comprising:
a plurality of beadlets, wherein each of the plurality of beadlets comprise:
a polymer beadlet shell, wherein creatine is dispersed within the polymer beadlet shell, the polymer beadlet shell comprises: gum Arabic; microcrystalline cellulose (MCC); hydroxypropyl methylcellulose (HPMC); glycerol; γ-cyclodextrin, wherein the γ-cyclodextrin comprises about 1-3 wt % of the polymer beadlet shell; ethyl cellulose; and one or more metal halide salt.
20. The composition of clause 19, wherein MCC comprises about 1-5 wt % of the polymer beadlet shell; wherein HPMC comprises about 1-5 wt % of the polymer beadlet shell; and wherein the ethyl cellulose comprises about 1-5 wt % of the polymer beadlet shell.
21. A method of providing a subject with sustained release of an active comprising: administering to the subject a composition comprising: a plurality of beadlets, wherein each of the plurality of beadlets comprise:
   a polymer beadlet shell, wherein creatine is dispersed within the polymer beadlet shell, the polymer beadlet shell comprises:
gum Arabic;
microcrystalline cellulose (MCC);
hydroxypropyl methylcellulose (HPMC);
glycerol;
γ-cyclodextrin, wherein the γ-cyclodextrin comprises about 1-3 wt % of the polymer beadlet shell; ethyl cellulose; and
one or more metal halide salt.
22. The method of clause 21, wherein the gum Arabic comprises from about 1-25 wt % of the polymer beadlet shell.
23. The method of clause 22, wherein MCC comprises about 1-5 wt % of the polymer beadlet shell.
24. The method of clause 23, wherein HPMC comprises about 1-5 wt % of the polymer beadlet shell.
25. The method of clause 21, wherein glycerol comprises about 1-3 wt % of the polymer beadlet shell.
26. The method of clause 21, wherein the γ-cyclodextrin comprises about 1-3 wt % of the polymer beadlet shell.
27. The method of clause 21, wherein the ethyl cellulose comprises about 1-5 wt % of the polymer beadlet shell.
28. The method of clause 21, wherein the one or more metal halide salt comprises sodium chloride.
29. The method of clause 28, wherein one or more metal halide salt further comprises potassium chloride.
30. The method of clause 29, wherein one or more metal halide salt further comprises magnesium chloride.
31. The method of any of clauses 21-30, wherein the one or more metal halide salt comprises from about 0.25-1 wt % of the polymer beadlet shell.
32. The method of clause 31, wherein one or more active is selected from beta-alanine, probiotics, caffeine, berberine and melatonin.
33. The method of any of clauses 21-31, wherein the active is creatine.
34. The method of clauses any of clauses 21-33, wherein the active is released over a period of from about 10-12 hours, following oral consumption.
35. The method of any of clauses 21-34, wherein each of the plurality of beadlets has a diameter of from about 500 to 2000 μm.
36. The method any of clauses 21-35, wherein the polymer beadlet shell further comprises a natural colorant.
37. The method of clause 36, wherein the natural colorant is selected from paprika extract, turmeric, beta-carotene, and beet extract.
38. The method of clause 37, wherein the natural colorant comprises from about 0.25 to 2.0 wt % of the polymer beadlet shell.

### EXPERIMENTAL

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1—Creatine Monohydrate Beadlets

Most skeletal muscles (˜95%) contain creatine, a naturally occurring guanidine molecule, although the brain, kidneys, liver, and testes also contain tiny amounts. Creatine can also be received exogenously from the diet, particularly in protein-rich meals like meat, fish, and nuts. Creatine is also synthesized in the liver and kidneys. The adenosine triphosphate (ATP)/phosphocreatine (PCr) phosphagen energy system is made up of creatine, which is essential for supplying an easily accessible source of phosphate for the resynthesis of ATP. Athletes and bodybuilders frequently utilize creatine dietary supplements, primarily in the form of creatine monohydrate (CM), as an ergogenic aid to improve activity and performance.

The creatine monohydrate content in a beadlet was analyzed by Agilent HPLC. Chromatographic separation was performed on C18 column at 25° C. The mobile phase is composed of 0.045 M Ammonium Sulphate in water, with a flow rate of 0.8 mL/min. A sample and an analytical standard were injected at a volume of 10 μl each. The detection wavelength for creatine monohydrate was 205 nm, and the retention time was 1 minute.

The concentration of creatine monohydrate in the samples was determined using the linear regression equation obtained from the respective standard calibration curves of creatine monohydrate (R2>0.999).

$$\% Creatine Monohydrate Loading = \frac{Amount of Creatine Monohydrate * 100}{Mass of Beadlets} \qquad Eq . 5$$

From the HPLC analysis it was found that the average creatine monohydrate content present in the beadlets was 75.2% with certain overages.

The release of Creatine monohydrate at physiological pH (1.2 or 6.8) was investigated using simulated intestinal fluid (SIF) prepared without enzymes according to USP42-NF37. The SIF was made up of 0.68 g of monobasic potassium phosphate dissolved in 25 mL of water, 7.7 mL of NaOH (0.2 M), and 100 mL of water. NaOH (1 M) was used to adjust the final pH to 6.8. The beadlets were immersed in 40 mL of SIF after being placed in a filtration bag. For 10 hours, the solution was mixed with a shaker. Samples were obtained every 30 minutes for the first two hours. The samples were then obtained every 1 hour for 10 hours. At each time interval, 3 mL of solution was withdrawn and replaced with 3 mL of fresh SIF.

The concentration of creatine monohydrate in the samples was determined using the respective calibration curves of creatine monohydrate in SIF (Y=68.512X+1538.2, R2=0.999). The cumulative release percentage of Creatine monohydrate from a creatine beadlet at time t (CR %) during in vitro dissolution was determined from the following equation.

$${D}_{( n )} = \frac{{A}_{t} * {W}_{s} * {V}_{D} * D L * {P}_{s} * 1000 * 100}{{A}_{s} * S D * {W}_{A} * 100 * {W}_{L}} \qquad Eq . 6$$

$${C}_{( n )} = \frac{{D}_{( n )} * {W}_{W}}{D {L}_{A}} \qquad Eq . 7$$

$$CR \% = {D}_{( n )} + C \qquad Eq . 8$$

$$C = {C}_{( n - 1 )} + {C}_{( n - 2 )} + {C}_{( n - X )} \qquad Eq . 9$$

In these equations “D(n)” is the drug release in percentage at a particular time; “AT” is the HPLC area of the test material; “WS” is the weight of the standard; “VD” is the weight of the dissolution medium; DL is the dilution; “PS” is the purity of the standard; “AS” is the HPLC area of the standard; SD is the standard dilution; “WA” is the total amount of creatine monohydrate in a beadlet; “WL” is the label claim of the creatine in a creatine beadlet; “C(n)” is the percentage content present in sample volume after particular time; “WW” is the weight of the withdrawn aliquot; “DLA” is the dissolution volume of the withdrawn sample; “C” is the sum of the percentage content present in sample volume after particular time: and “CR %” is the cumulative release percentage of creatine monohydrate from the beadlet at time t.

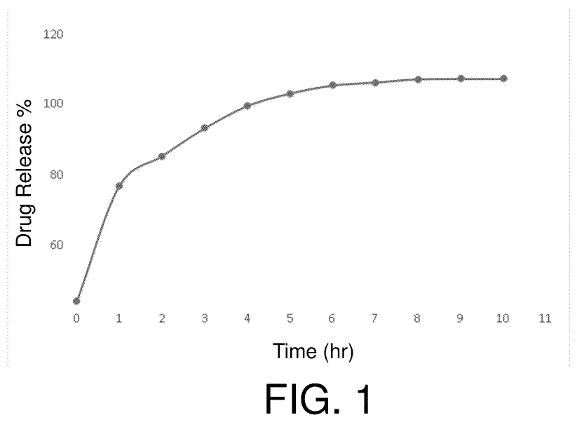

To obtain the rate of creatine monohydrate release in simulated intestinal fluid (SIF) from beadlets, with an initial weight of beadlets were incubated in 900 mL of SIF (pH 6.8) solutions with a consistent rotation for a period of 10 hours. The beadlets were dissolved in SIF within 1 hour. After 1 hour, a burst release of creatine monohydrate was observed and a release up to 99.9% was observed within 8 hours. The release of creatine monohydrate in S showed an initial release of 52% in the first hour and 85% in the next 4 hours, and an increase in release up to 95% after 6 hours followed by a slight release and reached a steady state at 8 hours. FIG. 1 shows a release curve.

Tables 1 & 2 show exemplary formulations of creatine monohydrate and creatine HCL beadlets, respectively.

TABLE 1
Creatine Monohydrate Beadlets

| | |
|---|---|
| Product Name | Creatine Monohydrate Beadlets |
| Date | May 2023 |

1. Physical Specification

| | |
|---|---|
| Appearance | Red Colou Spheres |
| Odour | Characteristics odour |
| Particle Size | NLT 80% passes through |
| | 14 mesh sieves |
| | NMT 20% passes through |
| | 20 mesh sieves |

2. Ingredients

| | |
|---|---|
| Active Ingredients | Specification |
| Creatine Monohydrate | 70-75% |
| Sodium Chloride | 0.25-0.35% |
| Potassium Chloride | 0.25-0.35% |
| Magnesium Citrate | 0.25-0.35% |
| Other Ingredients | |
| Gum Arabic | |
| MCC | |
| HPMC | |
| Glycerol | |
| y-Cyclodextrin | |
| Ethyl Cellulose | |
| Natural Colour | |
| (Paprika Extract) | |

TABLE 2
Creatine HCL Beadlets

| | |
|---|---|
| Product Name | Creatine HCL Beadlets |
| Date | May 2023 |

1. Physical Specification

| | |
|---|---|
| Appearance | Red Colou Spheres |
| Odour | Characteristics odour |
| Particle Size | NLT 80% passes through |
| | 14 mesh sieves |
| | NMT 20% passes through |
| | 20 mesh sieves |

2. Ingredients

| | |
|---|---|
| Active Ingredients | Specification |
| Creatine Monohydrate | 70-75% |
| Sodium Chloride | 0.25-0.35% |
| Potassium Chloride | 0.25-0.35% |
| Magnesium Citrate | 0.25-0.35% |
| Other Ingredients | |
| Gum Arabic | |
| MCC | |
| HPMC | |
| Glycerol | |
| y-Cyclodextrin | |
| Ethyl Cellulose | |
| Natural Colour | |
| (Paprika Extract) | |

### Example 2—Bioavailability of a Beaded Creatine Delivery System on Whole Body Creatine Retention

Background: Creatine monohydrate (CrM) is the used in the art to compare the efficacy of various other methods of creatine supplementation. The recommended loading dose of CrM is 4 doses of 5 grams per day for 5 to 7 days because oral ingestion of CrM peaks in the blood in about one hour and returns to baseline in about 4-5 hours. Ingesting several 5 g doses of CrM throughout the day promotes rapid creatine retention. While CrM loading protocols are effective, there has been interest in the art in determining ways to improve creatine retention. This Example examined whether providing CrM in micro-beads or beadlets that would delay the release of creatine into the blood over time will affect whole-body creatine retention during the first 24 hours after ingestion compared to CrM.

Methods: Fifteen females and males (28±6 years, 79.0±14 kg, 26.5±3 kg/m²) with no recent history of creatine supplementation participated in a double-blind, randomized, and crossover study with at least 7-days between testing sessions. Participants adhered to a plant-based protein diet prior to each testing session. Participants donated a pre-supplementation 24-hour urine sample. Participants were then randomly assigned to ingest capsules containing 5 g of CrM or 5 g of CrM prepared in beadlets (CrM-Bead). After 8 hours, participants then ingested another 5 g dose of the assigned supplement (total of 10 grams) and donated a 24-hour urine sample to assess whole-body creatine retention. Participants repeated the experiment after a seven-day washout while ingesting the crossover supplement, i.e. the CrM-Bead group was given CrM and the CrM group was given Crm-Beads. Whole-body creatine retention was determined by subtracting CrM intake from the difference between post-supplementation urine creatine content and expressed in percent retention.

Results: Analysis of whole-body creatine retention revealed an increase in 24-hour creatine retention with CrM-Bead supplementation compared to CrM (CrM 81.8%±11.4%, CrM-Bead 83.6%±12.5%). In summation, the percentage of creatine retention after 10 grams of Cr-Bead ingestion was higher compared with CrM.

Although the disclosure has been described with reference to preferred embodiments, persons skilled in the art will recognize that changes may be made in form and detail without departing from the spirit and scope of the disclosed apparatus, systems and methods

What is claimed is:

1. A composition for sustained release of one or more active comprising:
a plurality of beadlets, wherein each of the plurality of beadlets comprise:
a polymer beadlet shell, wherein the active is dispersed within the polymer beadlet shell, the polymer beadlet shell comprises:
gum Arabic;
microcrystalline cellulose (MCC);
hydroxypropyl methylcellulose (HPMC);
glycerol;
γ-cyclodextrin;
ethyl cellulose; and
one or more metal halide salt.

2. The composition of claim 1, wherein the gum arabic comprises from about 1-25 wt % of the polymer beadlet shell.

3. The composition of claim 2, wherein MCC comprises about 1-5 wt % of the polymer beadlet shell.

4. The composition of claim 3, wherein HPMC comprises about 1-5 wt % of the polymer beadlet shell.

5. The composition of claim 1, wherein glycerol comprises about 1-3 wt % of the polymer beadlet shell.

6. The composition of claim 1, wherein the γ-cyclodextrin comprises about 1-3 wt % of the polymer beadlet shell.

7. The composition of claim 1, wherein the ethyl cellulose comprises about 1-5 wt % of the polymer beadlet shell.

8. The composition of claim 1, wherein the one or more metal halide salt comprises sodium chloride.

9. The composition of claim 8, wherein one or more metal halide salt further comprises potassium chloride.

10. The composition of claim 9, wherein one or more metal halide salt further comprises magnesium chloride.

11. The composition of claim 1, wherein the one or more metal halide salt comprises from about 0.25-1 wt % of the polymer beadlet shell.

12. The composition of claim 1, wherein one or more active is selected from beta-alanine, probiotics, caffeine, berberine and melatonin.

13. The composition of claim 12, wherein the active is creatine.

14. The composition of claim 1, wherein the active is released over a period of from about 10-12 hours, following oral consumption.

15. The composition of claim 1, wherein each of the plurality of beadlets has a diameter of from about 500 to 2000 μm.

16. The composition of claim 1, wherein the polymer beadlet shell further comprises a natural colorant.

17. The composition of claim 16, the natural colorant is selected from paprika extract, turmeric, beta-carotene, and beet extract.

18. The composition of claim 17, wherein the natural colorant comprises from about 0.25 to 2.0 wt % of the polymer beadlet shell.

19. A composition for sustained release of creatine comprising:
a plurality of beadlets, wherein each of the plurality of beadlets comprise:
a polymer beadlet shell, wherein creatine is dispersed within the polymer beadlet shell, the polymer beadlet shell comprises:
gum arabic;
microcrystalline cellulose (MCC);
hydroxypropyl methylcellulose (HPMC);
glycerol;
γ-cyclodextrin, wherein the γ-cyclodextrin comprises about 1-3 wt % of the polymer beadlet shell;
ethyl cellulose; and
one or more metal halide salt.

20. A method of providing a subject with sustained release of one or more active comprising: administering to the subject a composition comprising: a plurality of beadlets, wherein each of the plurality of beadlets comprise:
a polymer beadlet shell, wherein creatine is dispersed within the polymer beadlet shell, the polymer beadlet shell comprises:
Gum Arabic;
microcrystalline cellulose (MCC);
hydroxypropyl methylcellulose (HPMC);
glycerol;
γ-cyclodextrin, wherein the γ-cyclodextrin comprises about 1-3 wt % of the polymer beadlet shell;
ethyl cellulose; and
one or more metal halide salt.
